(19) United States
(12) Patent Application Publication
(10) Pub. No.: US 2022/0398452 A1
(43) Pub. Date: Dec. 15, 2022

(54) SUPERVISED SIMILARITY LEARNING FOR COVARIATE MATCHING AND TREATMENT EFFECT ESTIMATION VIA SELF-ORGANIZING MAPS

(71) Applicants: International Business Machines Corporation, Armonk, NY (US); RENSSELAER POLYTECHNIC INSTITUTE, Troy, NY (US)

(72) Inventors: Dharmashankar Subramanian, White Plains, NY (US); Tian GAO, Berkeley Heights, NJ (US); Xiao Shou, Watervliet, NY (US); Kristin Paulette Bennett, Averill Park, NY (US)

(21) Appl. No.: 17/348,492

(22) Filed: Jun. 15, 2021

(51) Int. Cl. G06N 3/08; G06N 3/04; G16H 50/70; G16H 40/20; G16H 20/10

(52) U.S. Cl. CPC G06N 3/08; G06N 3/0481; G16H 50/70; G16H 40/20; G16H 20/10

(57) ABSTRACT

A classifying neural network (CNN) obtains a mixed data set of a priori information and outcomes information for treated units and untreated units. Classify units as treated or untreated, by running the CNN on the a priori information. Deliver a latent representation of the classified units from an intermediate layer of the CNN to a self-organizing map (SOM) engine. Generate an SOM based on the latent representation. Train the CNN to optimize a combined total loss of the classification and of the SOM. Estimate average treatment effect on the treated units by comparing the outcome information of the treated units to outcome information for untreated units that are nearest-neighbors of the treated units on the SOM.

### BACKGROUND

The present invention relates to the electrical, electronic, and computer arts, and more specifically, to artificial intelligence and machine learning.

It is desirable to apply machine learning to sets of “big data” or “mixed data” in which units of the sets incorporate many covariates (“high-dimensionality” data). This is desirable because comprehending such high-dimensionality data is beyond the capabilities of the human mind. However, in general, deriving actionable conclusions from high-dimensionality data also is beyond the capabilities of all or almost all extant computer systems (even including massively parallel multi-processor systems).

Referring to FIG. 1, for example, a typical data set 100 for an observational trial of a pharmaceutical compound (i.e., a trial in which participants are not randomly assigned to be treated or controlled, but rather are assigned treatment in response to an underlying condition) incorporates many units that differ along many dimensions (covariates). There are no two units that can be said to be identical but for their assignment to treatment or control. This makes it difficult to obtain values such as “ATT” (Average Treatment effect on the Treated), because predicting ATT requires matching covariates across units. Even for machine learning algorithms, it is prohibitively difficult to match covariates in order to predict ATT on big data sets.

### SUMMARY

Principles of the invention provide techniques for supervised similarity learning for covariate matching and treatment effect estimation via self-organizing maps. In one aspect, an exemplary method includes a classifying neural network obtaining a mixed data set that comprises a priori information and outcomes information for a plurality of treated units and a plurality of untreated units. The method also includes classifying one or more units of the mixed data set as treated or untreated, by running the classifying neural network on the a priori information for at least the one or more units. The method also includes delivering a latent representation of the classified units of the mixed data set from an intermediate layer of the classifying neural network to a self-organizing map engine. The method also includes generating a self-organizing map by the self-organizing map engine based on the latent representation. The method also includes training the classifying neural network to optimize a combined total loss of the classification and of the self-organizing map. The method also includes estimating average treatment effect on the treated units (“ATT”) by comparing the outcome information for at least one of the treated units to outcome information for an untreated unit that is a nearest-neighbor of the at least one treated unit on the self-organizing map. Advantageously, ATT can be estimated for data sets too big and too heterogeneous to admit of testing by conventional statistical methods.

Optionally, the method also includes A/B testing of the mixed data set based on comparison of outcome information for the treated units and for their nearest-neighbor counterfactuals. Advantageously, the A/B testing can be accomplished on data sets too big and too heterogeneous to admit of testing by conventional statistical methods.

Optionally, the method also includes making a treatment decision in response to the A/B testing based on the nearest-neighbor counterfactuals. Advantageously, such a treatment decision can be made on the basis of data too “big” for a human practitioner to fully comprehend without the assistance accorded by certain aspects of this invention.

One or more embodiments of the invention or elements thereof can be implemented in the form of a computer program product including a computer readable storage medium with computer usable program code for facilitating the method steps indicated. Furthermore, one or more embodiments of the invention or elements thereof can be implemented in the form of a system (or apparatus) including a memory that embodies computer executable instructions, and at least one processor that is coupled to the memory and operative by the instructions to facilitate exemplary method steps. Yet further, in another aspect, one or more embodiments of the invention or elements thereof can be implemented in the form of means for carrying out one or more of the method steps described herein; the means can include (i) hardware module(s), (ii) software module(s) stored in a tangible computer readable storage medium (or multiple such media) and implemented on a hardware processor, or (iii) a combination of (i) and (ii); any of (i)-(iii) implement the specific techniques set forth herein.

As used herein, “facilitating” an action includes performing the action, making the action easier, helping to carry the action out, or causing the action to be performed. Thus, by way of example and not limitation, instructions executing on one processor might facilitate an action carried out by instructions executing on a remote processor, by sending appropriate data or commands to cause or aid the action to be performed. For the avoidance of doubt, where an actor facilitates an action by other than performing the action, the action is nevertheless performed by some entity or combination of entities.

In view of the foregoing, techniques of the present invention can provide substantial beneficial technical effects. For example, one or more embodiments provide one or more of:

Simultaneously learning a meaningful latent representation and a corresponding metric with respect to a treatment assignment and an observational covariate space.

Automated selection for the size of latent dimensions with respect to an assignment by finding the minimum loss on a validation.

Improving the functionality of a computer as a tool for determining Average Treatment effect on the Treated (ATT).

Improving the functionality of a computer as a tool for estimating and visualizing counterfactuals to complex data sets.

Some embodiments may not have these potential advantages and these potential advantages are not necessarily required of all embodiments. These and other features and advantages of the present invention will become apparent from the following detailed description of illustrative embodiments thereof, which is to be read in connection with the accompanying drawings.

### BRIEF DESCRIPTION OF THE DRAWINGS

FIG. 1 depicts an excerpt of a typical big data set for an observational clinical trial.

FIG. 2 depicts a conceptual diagram of a self-organizing map.

FIG. 3 depicts a flowchart of a self-organizing map algorithm.

FIG. 4 depicts an architecture of a system for supervised similarity learning for covariate matching and treatment effect estimation via self-organizing maps, according to an exemplary embodiment.

FIG. 5 depicts steps of a method for implementing supervised similarity learning for covariate matching and treatment effect estimation via self-organizing maps, using the system of FIG. 4.

FIG. 6 depicts a cloud computing environment according to an embodiment of the present invention.

FIG. 7 depicts abstraction model layers according to an embodiment of the present invention.

FIG. 8 depicts a computer system that may be useful in implementing one or more aspects and/or elements of the invention, also representative of a cloud computing node according to an embodiment of the present invention.

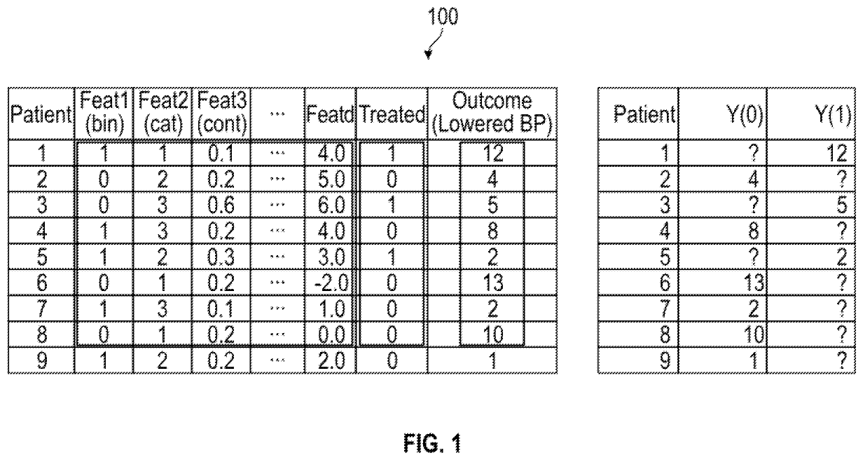

### DETAILED DESCRIPTION

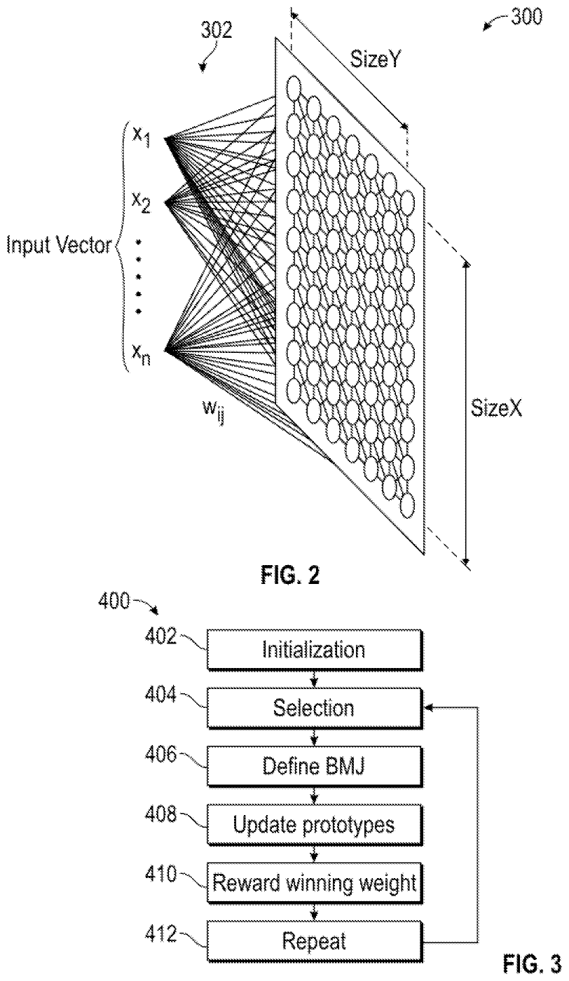

With reference to FIG. 2 and FIG. 3, a self-organizing map (SOM) 300 is a type of artificial neural network (ANN) that is trained using unsupervised learning to produce a low-dimensional (e.g., two-dimensional, three-dimensional, five-dimensional, seven-dimensional), discretized representation, called a map, of an input space of training samples 302. The SOM 300 therefore provides a method to carry out dimensionality reduction.

Generally, a neural network includes a plurality of computer processors that are configured to work together to implement one or more machine learning algorithms. The implementation may be synchronous or asynchronous. In a neural network, the processors simulate thousands or millions of neurons, which are connected by axons and synapses. Each connection is enforcing, inhibitory, or neutral in its effect on the activation state of connected neural units. Each individual neural unit has a summation function which combines the values of all its inputs together. In some implementations, there is a threshold function or limiting function on at least some connections and/or on at least some neural units, such that the signal must surpass the limit before propagating to other neurons. A neural network can implement supervised, unsupervised, or semi-supervised machine learning.

Self-organizing maps use a neighborhood function to preserve topological properties of the input space. FIG. 3 depicts an SOM online algorithm 400, according to an exemplary embodiment. The algorithm 400 includes, at 402, Initialization, in which weights for nodes are initialized. The weights of K nodes {1; 2 . . . K}, also called prototypes in SOM, are set as: mₖ (t), weight of node k at time t. In one or more embodiments, weights are initially random values between 0 and 1. At 404, the SOM algorithm 400 includes Selection, in which a vector x is chosen for training. In one or more embodiments, the vector x is randomly chosen. The term “unit” is used interchangeably for “vector” in portions of the following discussion. Then for each input vector x, at 406, a best match unit (BMU) c^{t}(x) is defined as the following:

c^{t}(x)=argmin_{kϵ{1, . . . ,K}}∥x−mₖ(t))∥²

At 408, the prototypes are then updated by assigning weights from the neighborhood of the BMU to neurons via

mₖ(t+1)=mₖ(t)+ϵ(t)h_{kc}_{^{t}}₍ₓ₎(t)(x−mₖ(t))

where ϵ(t) is the learning rate at t and h_{kc}_{^{t}}₍ₓ₎(t) is a neighborhood function. A Gaussian neighborhood function at time t is given as the following:

$${h}_{k {c}^{t} ( x )} = exp ( - \frac{{dist}^{2} ( k , l )}{2 {σ}^{2} ( t )} )$$

where dist² (k, l) is the Euclidean distance between k, l nodes on the map and σ(t) is the radius of influence at time t, which decays exponentially with respect to time t:

$$σ ( t ) = σ ( 0 ) \cdot {b}^{- \frac{t}{λ}}$$

where σ(0) is radius of influence at time 0, b is a logarithmic base, and λ is a time constant, i.e., total iterations. To achieve convergence, the learning rate ϵ is set:

$${\sum }_{t} ϵ ( t ) = + ∞$$

$${\sum }_{t} {ϵ ( t )}^{2} < + ∞$$

During mapping, one neuron will win: the neuron whose weight vector produces a result that lies closest to the input vector. This can be simply determined, at 410, by calculating a Euclidean distance between input vector and result of each weight vector. Then the matrix of weights is adjusted to be more like the winning neuron. Then at 412, repeat from 404 until total loss converges within a value selected by a practitioner. For example, a stopping condition may be that total loss changes by no more than 0.1% at subsequent iterations of training. Other values for a stopping condition will be apparent to an ordinary skilled worker, based on heuristics for the given application.

In a discrete setting, self-organizing mapping can be considered as a gradient descent process associated with

$$E ( m ) = \frac{1}{2 N} {\sum }_{i = 1}^{N} {\sum }_{k = 1}^{K} {h}_{k c ( {x}_{i} )} {‖ {m}_{k} - {x}_{i} ‖}^{2}$$

where E is an energy function or extended distortion of a stochastic process

m(t)=<m₁(t),m₂(t), . . . ,m_{K}(t)>^{T}

associated with the self-organizing map.

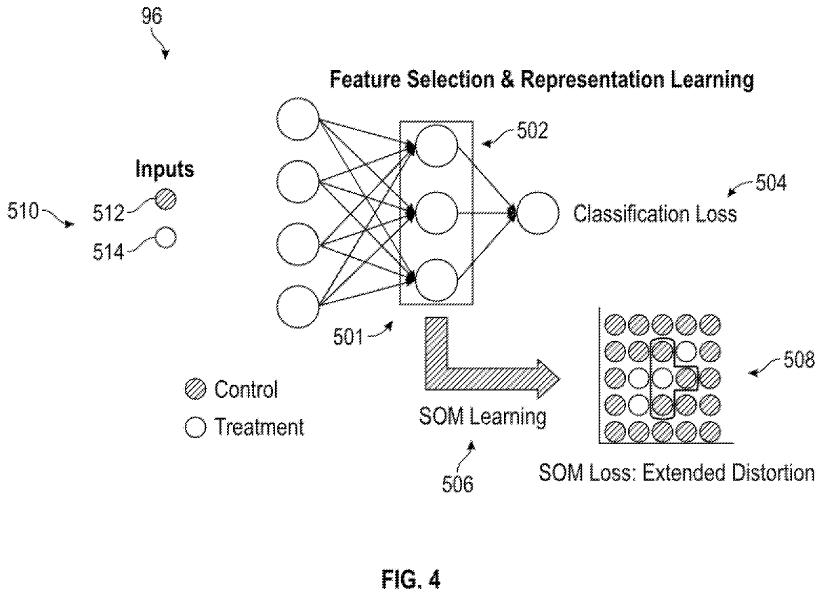

FIG. 4 depicts a system 96, which leverages the energy function E and implements a novel joint architecture that simultaneously learns a latent representation of input data and a corresponding “metric learning.” The system 96 incorporates a classifying neural network 501 for feature selection and representation learning. For each unit of input data 510, the classifying neural network 501 produces a classification 504 with an associated classification loss. The classifying neural network 501 incorporates an intermediate (hidden) layer 502; values of neurons in the intermediate layer 502 compose the aforementioned latent representation. The system 96 also incorporates an SOM engine 506, which produces an SOM 508 from the latent representation. The mixed data set 510, which is input to the system 96, includes control data 512 and treatment data 514. Each unit of data includes a priori information (e.g., for a medical application: demographics, physiology before medical treatment; for an industrial application: vibration of a machine before mechanical treatment) and outcome information (e.g., physiology after medical treatment, vibration of a machine after mechanical treatment by, e.g., changing viscosity of dampers supporting the machine).

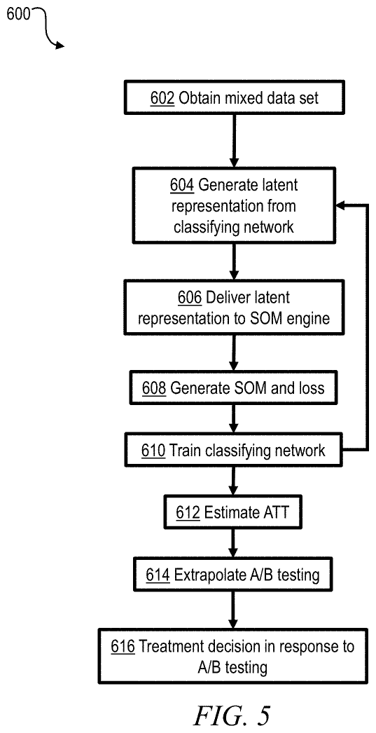

Overall, the system 96 is trained to implement a method 600 as depicted by FIG. 5. The method 600 includes several steps. At 602, obtain a mixed data set that includes a priori information and outcome information for a plurality of treated unit and a plurality of control (untreated) units. At 604, generate the latent representation by training the classifying neural network 501 to produce, from the a priori information, the classification 504 whether a given unit belongs to the treatment group or to the control group. During the classification training, at 606 deliver non-linearly transformed covariates (the latent representation) from the classifying neural network intermediate layer 502 to the self-organizing map engine 506 and, at 608, generate the SOM 508 and its corresponding loss. At 610, train the classifying neural network to optimize total loss of classification 504 and of the SOM 508. Then, at 612, estimate ATT by comparing the outcome information for nearest-neighbors on the SOM 508 that differ in their membership of control data 512 or treatment data 514. In one or more embodiments, at 614, extrapolate AB testing from comparison of outcome information for the treated units and for their nearest-neighbor counterfactuals.

Further details of the method 600 are presented with reference to a total loss for training the system 96, which is given as:

Lₜₒₜ(W,m,c)=L_{BCE}(W)γL_{SOM}(W,m,c)

where W represents the set of weights and biases in the classifying neural network, m is the set of weight vectors in the self-organizing map, c is short for c^{t}(x), the argmin function that finds the BMU in the self-organizing map, and γ is a weighting factor for trade-off between the two losses. L_{BCE}, which is used for training feature selection and representation learning 502, is the familiar binary cross-entropy loss, which is standard for classification tasks. In this case, the classification is between “Treatment” or “Control” categories for each member of the data set. The loss for each member is binary, either 0 for a match between prediction and actual or 1 for a contradiction. Explicitly defined,

$${L}_{B C E} ( W ) = - {\sum }_{i = 1}^{N} {T}_{i} log ( sigmoid ( {f}_{W} ( {x}_{i} ) ) ) + ( 1 - {T}_{i} ) log ( 1 - sigmoid ( {f}_{W} ( {x}_{i} ) ) )$$

where Tᵢ is yes or no on treatment for unit i, and f_{W}(xᵢ) is a nonlinearly transformed version (latent representation) of xᵢ, the covariates of unit i. In other words, f_{W}(xᵢ) represents the values of neurons in an intermediate layer of the classifying neural network used for classification.

Similarly, self-organizing map (SOM) loss for the system 96 can be defined

$${L}_{S O M} = {\sum }_{i = 1}^{N} {\sum }_{k = 1}^{K} {h}_{k {c}^{t} ( {f}_{W} ( {x}_{i} ) )} {‖ {m}_{k} - {f}_{w} ( {x}_{i} ) ‖}^{2}$$

with t as the iteration number, m is the set of weight vectors in the self-organizing map, h_{kc}(t) is a Gaussian kernel, and

c^{t}(x)=argminₖ∥f_{W}(xᵢ)(t)−mₖ(t)∥.

BCE loss, SOM loss, and total loss are updated at each iteration. Note that c^{t}(x), the argmin function at iteration t that produces the BMU for each unit xᵢ of input data, is non-differentiable; accordingly, in one or more embodiments it is fixed at each optimization step so that back-propagation continues easily.

Thus, the intermediate layer of the classifying neural network feeds the SOM engine. This means that dimensionality of the inputs to the SOM is reduced from the full range of covariates, while the classifying neural network preserves higher-level features (the latent representation) that incorporate the physical significance of the covariates. As a result, the SOM problem becomes tractable for high-dimensionality covariates, whereas before it was not computationally feasible to generate an SOM for such a large number of covariates as would be present in a typical clinical trial. More generally, aspects of the invention are applicable to any situation in which it is desirable to estimate counterfactuals for large data sets with a great number of covariates (e.g., in one or more embodiments more than 10 variables, in other embodiments more than 20 variables). Certain aspects of the invention are particularly applicable to estimation of binary counterfactuals, e.g., “Treatment” vs. “Control” or “Powered” vs. “Not Powered.”

Using the system 96, Average Treatment effect on the Treated (ATT) can be obtained by solving the classification subproblem with respect to treated or not treated; delivering the latent representation from the classifying neural network intermediate layer 502 to the self-organizing map engine 506; generating the SOM 508; and training the classifier network 501 to optimize total loss of the classification and of the SOM. When learning is complete, the system 96 maps input data 510 (Xϵ [symbol] ^{N×d}) through the intermediate layer 502 of the classifying neural network 501 to the self-organizing map 508, which is of smaller dimensions than the input data 510 (e.g., two dimensions, three dimensions, five dimensions, or seven dimensions). Thus, for each treatment unit, a similar unit of control is matched as a nearest neighbor on the SOM 508. Then at 612, ATT can be estimated by comparing outcome information across the two nearest neighbors (one a member of control data 512, the other a member of treatment data 514).

Once ATT is estimated, a practitioner can determine whether to treat a control unit in view of ATT, which includes potential side effects or other risks of treatment. Exemplary modes of treatment for various units include:

Supplying or withholding a pharmacological treatment for a patient with a medical condition.

Enrolling or not enrolling an insurance plan participant in a complex care management program.

Supplying or not supplying a wearable fitness tracker to a participant in an insurance plan.

Approving or withholding approval for a vaccination.

Authorizing or withholding advertising spend on a proposed campaign.

Delivering or not delivering a smart home control device to a utility consumer.

Supplying or not supplying a whole-house water filter to a utility consumer.

Given the discussion thus far, and with reference to the accompanying drawings, it will be appreciated that, in general terms, an exemplary method 600, according to an aspect of the invention, includes, at 602, a classifying neural network obtaining a mixed data set that comprises a priori information and outcomes information for a plurality of treated units and a plurality of untreated units. The method 600 also includes, at 604, classifying one or more units of the mixed data set as treated or untreated (and generate a latent representation of the classified units), by running the classifying neural network on the a priori information for at least the one or more units. At 606, deliver the latent representation of the classified units of the mixed data set from an intermediate layer of the classifying neural network to a self-organizing map engine. At 608, generate a self-organizing map by the self-organizing map engine based on the latent representation. At 610, train the classifying neural network to optimize a combined total loss of the classification and of the self-organizing map. At 612, estimate average treatment effect on the treated units by comparing the outcome information for at least one of the treated units to outcome information for an untreated unit that is a nearest-neighbor of the at least one treated unit on the self-organizing map.

In one or more embodiments, the method 600 also includes, at 614, A/B testing of the mixed data set based on comparison of outcome information for the treated units and for their nearest-neighbor counterfactuals.

In one or more embodiments, the method includes at 616 making a treatment decision in response to the A/B testing based on the nearest-neighbor counterfactuals. For example: enrolling an insurance plan participant into a complex care management program; supplying a wearable fitness tracker to an insurance plan participant; approving a vaccine for delivery to the general population; authorizing advertising spend on a new campaign; supplying a smart home control device to a utility consumer; administering a pharmacological treatment to a clinical patient.

In one or more embodiments, the method 600 includes calculating loss of the classification L_{BCE} as L_{BCE}(W)=−Σᵢ₌₁^{N} Tᵢ log(sigmoid(f_{W}(xᵢ)))+(1−Tᵢ) log(1−sigmoid(f_{W}(xᵢ))), where W represents the matrix of weights and biases in the classifying neural network, Tᵢ is yes or no on treatment for unit i, and f_{W}(xᵢ) is a latent representation of xᵢ, the covariates of unit i.

In one or more embodiments, the method 600 includes calculating loss of the self-organizing map L_{SOM} as L_{SOM}=Σᵢ₌₁^{N} Σₖ₌₁^{K} h_{kc}_{^{t}}(f_{W}(xᵢ))∥mₖ−f_{W}(xᵢ)∥² where t is the iteration number, f_{W}(xᵢ) is a latent representation of covariates xᵢ of unit i, m is the set of weight vectors in the self-organizing map, h_{kc}(t) is a Gaussian kernel, and

c^{t}(x)=argminₖ∥f_{W}(xᵢ)(t)−mₖ(t)∥.

One or more embodiments of the invention, or elements thereof, can be implemented in the form of an apparatus including a memory and at least one processor that is coupled to the memory and operative to perform exemplary method steps, or in the form of a non-transitory computer readable storage medium embodying computer executable instructions which when executed by a computer cause the computer to perform exemplary method steps.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

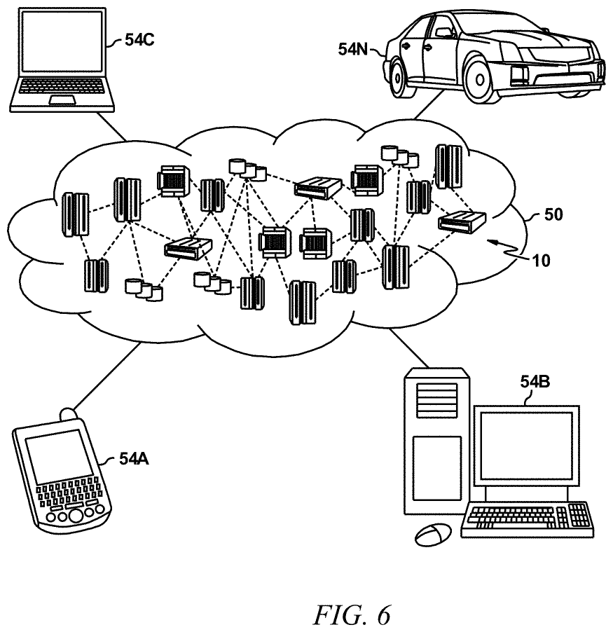

Referring now to FIG. 6, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 6 are intended to be illustrative only and that computing nodes 10 and cloud computing environment 50 can communicate with any type of computerized device over any type of network and/or network addressable connection (e.g., using a web browser).

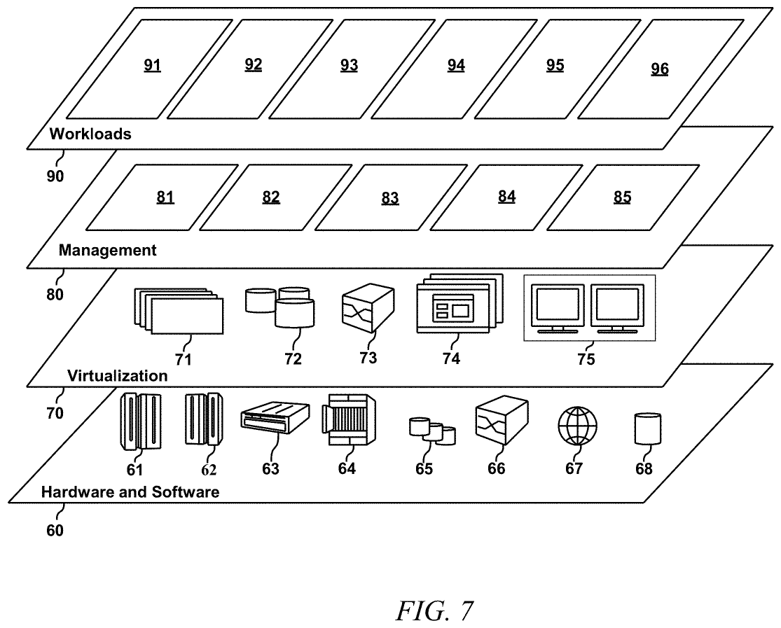

Referring now to FIG. 7, a set of functional abstraction layers provided by cloud computing environment 50 (FIG. 6) is shown. It should be understood in advance that the components, layers, and functions shown in FIG. 7 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and at least a portion of a combined classifier and self-organizing map 96 that implements supervised similarity learning for covariate matching and treatment effect estimation via self-organizing maps.

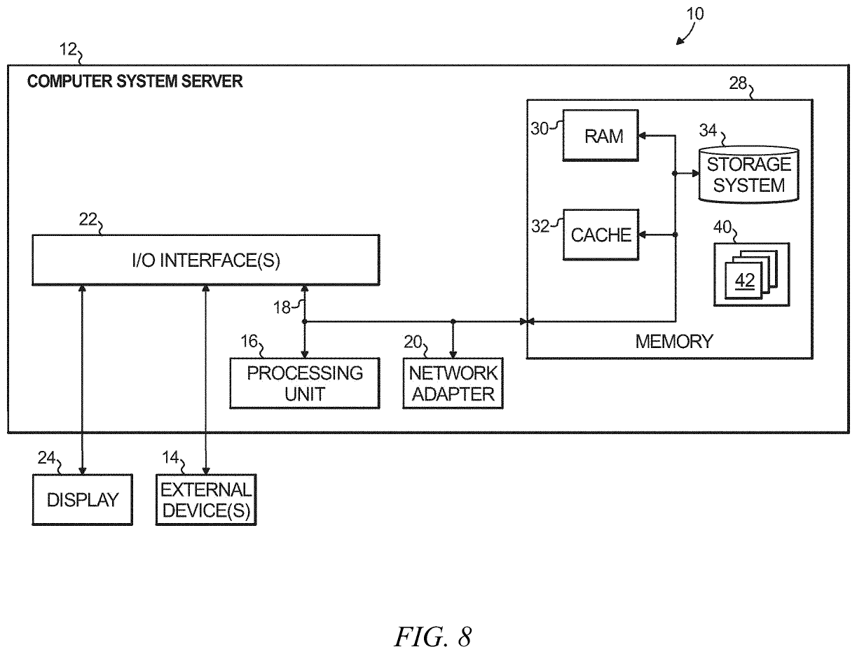

FIG. 8 depicts a computer system that may be useful in implementing one or more aspects and/or elements of the invention, also representative of a cloud computing node according to an embodiment of the present invention. Referring now to FIG. 8, cloud computing node 10 is only one example of a suitable cloud computing node and is not intended to suggest any limitation as to the scope of use or functionality of embodiments of the invention described herein. Regardless, cloud computing node 10 is capable of being implemented and/or performing any of the functionality set forth hereinabove.

In cloud computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 8, computer system/server 12 in cloud computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, and external disk drive arrays, RAID systems, tape drives, and data archival storage systems, etc.

Thus, one or more embodiments can make use of software running on a general purpose computer or workstation. With reference to FIG. 8, such an implementation might employ, for example, a processor 16, a memory 28, and an input/output interface 22 to a display 24 and external device(s) 14 such as a keyboard, a pointing device, or the like. The term “processor” as used herein is intended to include any processing device, such as, for example, one that includes a CPU (central processing unit) and/or other forms of processing circuitry. Further, the term “processor” may refer to more than one individual processor. The term “memory” is intended to include memory associated with a processor or CPU, such as, for example, RAM (random access memory) 30, ROM (read only memory), a fixed memory device (for example, hard drive 34), a removable memory device (for example, diskette), a flash memory and the like. In addition, the phrase “input/output interface” as used herein, is intended to contemplate an interface to, for example, one or more mechanisms for inputting data to the processing unit (for example, mouse), and one or more mechanisms for providing results associated with the processing unit (for example, printer). The processor 16, memory 28, and input/output interface 22 can be interconnected, for example, via bus 18 as part of a data processing unit 12. Suitable interconnections, for example via bus 18, can also be provided to a network interface 20, such as a network card, which can be provided to interface with a computer network, and to a media interface, such as a diskette or CD-ROM drive, which can be provided to interface with suitable media.

Accordingly, computer software including instructions or code for performing the methodologies of the invention, as described herein, may be stored in one or more of the associated memory devices (for example, ROM, fixed or removable memory) and, when ready to be utilized, loaded in part or in whole (for example, into RAM) and implemented by a CPU. Such software could include, but is not limited to, firmware, resident software, microcode, and the like.

A data processing system suitable for storing and/or executing program code will include at least one processor 16 coupled directly or indirectly to memory elements 28 through a system bus 18. The memory elements can include local memory employed during actual implementation of the program code, bulk storage, and cache memories 32 which provide temporary storage of at least some program code in order to reduce the number of times code must be retrieved from bulk storage during implementation.

Input/output or I/O devices (including but not limited to keyboards, displays, pointing devices, and the like) can be coupled to the system either directly or through intervening I/O controllers.

Network adapters 20 may also be coupled to the system to enable the data processing system to become coupled to other data processing systems or remote printers or storage devices through intervening private or public networks. Modems, cable modem and Ethernet cards are just a few of the currently available types of network adapters.

As used herein, including the claims, a “server” includes a physical data processing system (for example, system 12 as shown in FIG. 8) running a server program. It will be understood that such a physical server may or may not include a display and keyboard.

One or more embodiments can be at least partially implemented in the context of a cloud or virtual machine environment, although this is exemplary and non-limiting. Reference is made back to FIG. 6 and FIG. 7 and accompanying text.

It should be noted that any of the methods described herein can include an additional step of providing a system comprising distinct software modules embodied on a computer readable storage medium; the modules can include, for example, any or all of the appropriate elements depicted in the block diagrams and/or described herein; by way of example and not limitation, any one, some or all of the modules/blocks and or sub-modules/sub-blocks described. The method steps can then be carried out using the distinct software modules and/or sub-modules of the system, as described above, executing on one or more hardware processors such as 16. Further, a computer program product can include a computer-readable storage medium with code adapted to be implemented to carry out one or more method steps described herein, including the provision of the system with the distinct software modules.

Exemplary System and Article of Manufacture Details

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method comprising:
obtaining by a classifying neural network a mixed data set that comprises a priori information and outcomes information for a plurality of treated units and a plurality of untreated units;
classifying one or more units of the mixed data set as treated or untreated, by running the classifying neural network on the a priori information for at least the one or more units;
delivering a latent representation of the classified units of the mixed data set from an intermediate layer of the classifying neural network to a self-organizing map engine;
generating a self-organizing map by the self-organizing map engine based on the latent representation;
training the classifying neural network to optimize a combined total loss of the classification and of the self-organizing map; and
estimating average treatment effect on the treated units by comparing the outcome information for at least one of the treated units to outcome information for an untreated unit that is a nearest-neighbor of the at least one treated unit on the self-organizing map.

2. The method of claim 1, further comprising A/B testing of the mixed data set based on comparison of outcome information for the treated units and for their nearest-neighbor counterfactual s.

3. The method of claim 2, further comprising enrolling an insurance plan participant into a complex care management program in response to the A/B testing based on the nearest-neighbor counterfactual s.

4. The method of claim 2, further comprising supplying a wearable fitness tracker to an insurance plan participant in response to the A/B testing based on the nearest-neighbor counterfactual s.

5. The method of claim 2, further comprising approving a vaccine for delivery to the general population in response to the A/B testing based on the nearest-neighbor counterfactuals.

6. The method of claim 2, further comprising authorizing advertising spend on a new campaign in response to the A/B testing based on the nearest-neighbor counterfactuals.

7. The method of claim 2, further comprising supplying a smart home control device to a utility consumer in response to the A/B testing based on the nearest-neighbor counterfactuals.

8. The method of claim 2, further comprising administering a pharmacological treatment to a clinical patient in response to the A/B testing based on the nearest-neighbor counterfactuals.

9. The method of claim 1, further comprising calculating loss of the classification L_{BCE} as
$${L}_{B C E} ( W ) = - {\sum }_{i = 1}^{N} {T}_{i} log ( sigmoid ( {f}_{W} ( {x}_{i} ) ) ) + ( 1 - {T}_{i} ) log ( 1 - sigmoid ( {f}_{W} ( {x}_{i} ) )$$
where W represents the matrix of weights and biases in the classifying neural network, Tᵢ is yes or no on treatment for unit i, and f_{W}(xᵢ) is a latent representation of xᵢ, the covariates of unit i.

10. The method of claim 1, further comprising calculating loss of the self-organizing map L_{SOM} as
$${L}_{S O M} = {\sum }_{i = 1}^{N} {\sum }_{k = 1}^{K} {h}_{k {c}^{t} ( {f}_{W} ( {x}_{i} ) )} {‖ {m}_{k} - {f}_{w} ( {x}_{i} ) ‖}^{2}$$
where t is the iteration number, f_{W}(xᵢ) is a latent representation of covariates xᵢ of unit i, m is the set of weight vectors in the self-organizing map, h_{kc}(t) is a Gaussian kernel, and
c^{t}(x)=argminₖ∥f_{W}(xᵢ)(t)−mₖ(t)∥.

11. A computer program product comprising one or more computer readable storage media that embody computer executable instructions, which when executed by a computer cause the computer to perform a method comprising:
obtaining, by a classifying neural network, a mixed data set that comprises a priori information and outcomes information for a plurality of treated units and a plurality of untreated units;
classifying one or more units of the mixed data set as treated or untreated, by running the classifying neural network on the a priori information for at least the one or more units;
delivering a latent representation of the classified units of the mixed data set from an intermediate layer of the classifying neural network to a self-organizing map engine;
generating a self-organizing map by the self-organizing map engine based on the latent representation;
training the classifying neural network to optimize a combined total loss of the classification and of the self-organizing map; and
estimating average treatment effect on the treated units among the one or more units by comparing outcome information for at least one treated unit of the one or more units to outcome information for an untreated unit of the one or more units that is a nearest-neighbor on the self-organizing map to the at least one treated unit.

12. The computer program product of claim 11, wherein the method further comprises:
AB testing of the mixed data set based on comparison of outcome information for the treated units and for their nearest-neighbor counterfactuals.

13. The computer program product of claim 11, wherein the method further comprises:
calculating loss of the classification L_{BCE} as
$${L}_{B C E} ( W ) = - {\sum }_{i = 1}^{N} {T}_{i} log ( sigmoid ( {f}_{W} ( {x}_{i} ) ) ) + ( 1 - {T}_{i} ) log ( 1 - sigmoid ( {f}_{W} ( {x}_{i} ) ) )$$
where W represents the matrix of weights and biases in the classifying neural network, Tᵢ is yes or no on treatment for unit i, and f_{W}(xᵢ) is a latent representation of xᵢ, the covariates of unit i.

14. The computer program product of claim 11, wherein the method further comprises:
calculating loss of the self-organizing map L_{SOM} as
$${L}_{S O M} = {\sum }_{i = 1}^{N} {\sum }_{k = 1}^{K} {h}_{{kc}^{t} ( {f}_{W} ( {x}_{i} ) )} {‖ {m}_{k} - {f}_{W} ( {x}_{i} ) ‖}^{2}$$
where t is the iteration number, f_{W}(xᵢ) is a latent representation of covariates xᵢ of unit i, m is the set of weight vectors in the self-organizing map, h_{kc}(t) is a Gaussian kernel, and
c^{t}(x)=argminₖ∥f_{W}(xᵢ)(t)−mₖ(t)∥.

15. An apparatus comprising:
a memory embodying computer executable instructions; and
at least one processor, coupled to the memory, and operative by the computer executable instructions to perform a method comprising:
obtaining, by a classifying neural network, a mixed data set that comprises a priori information and outcomes information for a plurality of treated units and a plurality of untreated units;
classifying one or more units of the mixed data set as treated or untreated, by running the classifying neural network on the a priori information for at least the one or more units;
delivering a latent representation of the classified units of the mixed data set from an intermediate layer of the classifying neural network to a self-organizing map engine;
generating a self-organizing map by the self-organizing map engine based on the latent representation;
training the classifying neural network to optimize a combined total loss of the classification and of the self-organizing map; and
estimating average treatment effect on the treated units among the one or more units by comparing outcome information for at least one treated unit of the one or more units to outcome information for an untreated unit of the one or more units that is a nearest-neighbor on the self-organizing map to the at least one treated unit.

16. The apparatus of claim 15, wherein the method further comprises:
calculating loss of the classification L_{BCE} as
$${L}_{B C E} ( W ) = - {\sum }_{i = 1}^{N} {T}_{i} log ( sigmoid ( {f}_{W} ( {x}_{i} ) ) ) + ( 1 - {T}_{i} ) log ( 1 - sigmoid ( {f}_{W} ( {x}_{i} ) ) )$$
where W represents the matrix of weights and biases in the classifying neural network, Tᵢ is yes or no on treatment for unit i, and f_{W}(xᵢ) is a latent representation of xᵢ, the covariates of unit i.

17. The apparatus of claim 15, wherein the method further comprises:
calculating loss of the self-organizing map L_{SOM} as
$${L}_{S O M} = {\sum }_{i = 1}^{N} {\sum }_{k = 1}^{K} {h}_{k {c}^{t} ( {f}_{W} ( {x}_{i} ) )} {‖ {m}_{k} - {f}_{W} ( {x}_{i} ) ‖}^{2}$$
where t is the iteration number, f_{W}(xᵢ) is a latent representation of covariates xᵢ of unit i, mₖ is the set of weight vectors in the self-organizing map, h_{kc}(t) is a Gaussian kernel, and
c^{t}(x)=argminₖ∥f_{W}(xᵢ)(t)−mₖ(t)∥.

18. The apparatus of claim 15, wherein the method further comprises:
A/B testing of the mixed data set based on comparison of outcome information for the treated units and for their nearest-neighbor counterfactuals.

19. The apparatus of claim 18, wherein the method further comprises:
supplying a smart home control device to a utility consumer in response to the A/B testing based on the nearest-neighbor counterfactuals.

20. The apparatus of claim 18, wherein the method further comprises:
administering a pharmacological treatment to a clinical patient in response to the A/B testing based on the nearest-neighbor counterfactuals.
